(19) United States
(12) Plant Patent Application Publication
(10) Pub. No.: US 2017/0013762 P1
(43) Pub. Date: Jan. 12, 2017

(54) Vanilla variety named 'handa'

(50) ;

(71) Applicant: French Agricultural Research Centre for International Development ( CIRAD), Saint - Pierre (FR)

(72) Inventors: Michel Grisoni, Saint-Pierre (FR); Jean-Bernard Dijoux, Saint-Pierre (FR)

(21) Appl. No.: 14/999,830

(22) Filed: Jul. 6, 2016

(30) Foreign Application Priority Data
Jul. 7, 2015 (EP) 20151561

(51) Int. Cl. A01H 5/00

(52) U.S. Cl. PLT/311; PLT/258

(57) ABSTRACT

A new vanilla variety (Vanilla planifolia, vanilla orchid) designated as ‘Handa’ is provided that produces aromatic fruit having a suitable vanillin content and combines superior agronomic and technologic properties as compared to ‘Ordinary’ vanilla, notably resistance to Fusarium oxysporum f. sp. radicis vanillae, and indehiscence of the fruit.

### CROSS-REFERENCE TO RELATED APPLICATION

This application claims the benefit, under 35 U.S.C. 119, of Community Plant Variety Office (CPVO) Application No. 2015/1561, filed Jul. 7, 2015; which is incorporated herein by reference in its entirety.

### LATIN NAME

Botanical/commercial classification: (Vanilla planifolia), new vanilla cultivar.

### VARIETAL DENOMINATION

The varietal denomination of the claimed vanilla variety is ‘Handa’.

### BACKGROUND OF THE INVENTION

Commercial vanilla is produced by curing the mature fruits of Vanilla planifolia or V.×tahitensis (Orchidaceae). The largest portion of the vanilla marketed worldwide comes from V. planifolia and originates from the Indian Ocean region. Madagascar provides over 70% of the world's vanilla production, with the vanilla coming from small village plantations in the Northern provinces of this country. Vanilla is also produced in e.g. Indonesia (10%), Comoros (5%), India (5%), and Uganda (5%). The United States of America, Europe, and Japan are the main vanilla consumer countries. However, current world production of vanilla, which is about 2,500 tons/year, only partially meets the annual global demand for vanilla, which is estimated at more than 3,000 tons/year. This deficiency in vanilla supply is typically met by production of vanillin through synthesis or biosynthesis from various raw materials in order to accommodate demand.

Vanilla planifolia originated in Mexico and Central America. Some genetic diversity still exists in protected natural areas and farmer plots, but this diversity tends to diminish year after year (Soto Arenas 1999; Soto Arenas 2003). From its native region, very few vanilla varieties are cultivated and none of which have been accurately characterized (Roux-Cuvelier & Grisoni 2010). Global vanilla production is therefore based on an extremely narrow genetic base, which limits the possibilities of adapting to global changes (e.g. global climate change). The premature fruit drop that dramatically impacts Mexican farmers in the Veracruz region is considered a harbinger of the impact of climate change on this traditional crop (Hernandez Hernandez 2011). In the Indian Ocean area, the increasing pressure of pests and diseases in production plots strongly hampers the vanilla industry (Grisoni et al. 2009). In particular, the root rot disease, caused by the soil borne fungus Fusarium oxysporum f. sp. radicis vanillae (Koyyappurath et al. 2015), has had severe impacts on the vanilla market since the 2000s.

Obtaining new genetic resources for vanilla, which are e.g. better adapted to the biotic and abiotic constraints while still meeting commercial requirements, is therefore an issue of primary importance for the global vanilla market and vanilla breeders. Indeed, vanilla remains one of the most requested and popular spices in the world. Accordingly, there exists a need for improved vanilla varieties with improved disease resistance and commercial viability as compared to other traditional vanilla varieties.

### BRIEF SUMMARY

The present invention relates to a new and distinct vanilla cultivar (Vanilla planifolia) that has been denominated as ‘Handa’, and more particularly as a vanilla variety that produces aromatic fruit having a suitable vanillin content and combines superior agronomic and technologic properties as compared to ‘Ordinary’ vanilla, notably resistance to Fusarium oxysporum f. sp. radicis vanillae, and indehiscence of the fruit.

Applicant developed a breeding program for vanilla aimed at producing vanilla plants with disease resistance and having favorable agronomic properties. The high heterozygosity of vanilla clones inherited from their natural sexual reproduction that favors outcrossing (Soto Arenas 1999) suggested that new vanilla varieties could be recovered by recombination of characters in self-pollinated progenies. Applicant's vanilla breeding program involved self-pollination of the ‘Ordinary’ vanilla variety and selection of progeny with desirable characteristics. The present vanilla variety ‘Handa’ as described herein is a product of these breeding efforts, as outlined below.

In November of Year 1, an ‘Ordinary’ vanilla plant (Vanilla planifolia) being cultivated at CIRAD La Bretagne (La Réunion, France) was manually auto-pollinated. Progeny seeds resulting from this self-pollination were collected from a mature fruit in June of Year 2, and these seeds were plated on Murashige and Skoog (MS) culture media (Murashige & Skoog 1962). Between February and May of Year 3, the germinated seeds (protocorms) were individually subcultured onto new MS media in tubes. Regular subculture of the 11 vanilla seedling lines recovered from the initial plates (accession numbers CR0019 to CR0029) took place from Year 4-Year 9. In Year 9, these 11 vanilla seedlings were transferred to the Plant Protection Platform (CIRAD, Saint Pierre) for conservation and acclimatization of the material. In Year 12, the acclimatized lines were planted into the shadehouse of CRB VATEL (La Réunion, France) for field evaluation. The vanilla seedling designated CR0020 was selected and is presented herein as variety ‘Harada’.

From Year 4 to Year 9, vanilla variety ‘Handa’ was maintained in vitro, and was asexually reproduced by micro-cuttings every 10^{th} month, on MS medium, in CIRAD's laboratory in La Bretagne (La Réunion, France). From Year 9 to the present, vanilla variety ‘Handa’ has been reproduced by micro-cuttings in vitro, on MS medium, at the CIRAD laboratory of Saint Pierre, (La Réunion, France). After acclimatization of vanilla variety ‘Handa’ in Year 12, this variety was also asexually reproduced by cuttings and cultivated in the shadehouse of CRB VATEL facilities in Saint Pierre. Between Year 18 and Year 20, vanilla variety ‘flanda’ was planted in three experimental plots of La Réunion for a multilocal trial in the municipalities of Sainte Rose, Saint Philippe, and Saint André.

### DESCRIPTION OF THE FIGURES

FIG. 1 illustrates vines of vanilla variety ‘Handa’ bearing young fruit.

FIG. 2 illustrates a flower of vanilla variety ‘Handa’.

FIG. 3 illustrates green fruit (left) and fully mature, non-dehiscent fruit (right) of vanilla variety ‘Handa’.

### BOTANICAL DESCRIPTION OF THE PLANT

The following is a detailed botanical description of the new vanilla variety designated as ‘Handa’, including the key differentiating characteristics of this variety and comparisons of certain characteristics of ‘Handa’ to other vanilla varieties.

Vanilla planifolia Jacks (ex Andrews), commonly called vanilla, is a hemi-epiphytic orchid vine that is native to Mexico and Central America. In its native habitat (rain forest), it may grow up to 10-30 m tall over time. The vine has a thick, fleshy stem (5 to 15 mm diameter) bearing alternate, succulent, flat-bladed elliptic leaves (20 ×15 cm L×1, 1-2 mm thick) and adventitious, aerial roots, which allow it to cling to its support. The leaves and stem are pale to dark green depending on light intensity. The vine generally produces greenish flowers once per year (about 5 cm wide) that open early in the morning and close in the afternoon. Due to a membrane called the rostellum that forms a physical barrier between the male and female organs in the flower, fertilization of the ovules by pollen should be assisted by a pollinator. In nature, pollination is accomplished by bees. In culture, vanilla flowers need to be hand pollinated in order to produce fruits. The fruits (15-23 cm long, 0.8-1.6 cm wide) reach maturity 8-10 months after pollination. A post-harvest curing of the fruits during 4 to 9 months is necessary to obtain the stable, aromatic and dry vanilla pods that constitute the commercial vanilla.

Since vanilla vines may be reproduced by cuttings (80-120 cm long stem fragment), they have been clonally propagated throughout the world by this method, and from few initial materials collected in Mexico (Bory et al. 2008b). As a consequence, the vast majority of the cultivated vanilla belong to the ‘Ordinary’ type, also called ‘Classique’ in Reunion Island, ‘Mansa’ in Mexico, and ‘Gasy’ in Madagascar. However, a few natural variants of Vanilla planifolia detected in farmers' plots have been described (Castillo Martinez & Engleman 1993; Bory et al. 2008a). Table 1 summarizes the main distinctive traits of these varieties.

TABLE 1
Comparison of vanilla varieties to ‘Ordinary’ vanilla

| Variety | Distinctiveness as compared to ‘Ordinary’ vanilla |
|---|---|
| Aiguille | Lanceolate leaves, thin fruit and low vigor. |
| Mexique | Slightly bigger fruits with rounded section, |
| | darker leaf color. |
| Variegata or | Presence of white/yellow stripes on leaf, stem, fruit. |
| Rayada | |
| Grosse vanille | Bigger and thicker leaves, bigger stem and flower |
| | (tetraploid), indehiscent fruit. |
| Male sterile | Abortion of auto-pollinated fruits (triploid). |
| Oreja de burro | Leaf, stem and flower are bigger, early blooming, |
| | abortion of auto-pollinated fruits. |

Interspecific hybrids have also been produced in Puerto Rico, Madagascar, and India (Theis & Jimenez 1957; Delassus 1963; Minoo et al. 2006). These plants have genetic, morphologic, and aromatic properties very distinct from those of V. planifolia.

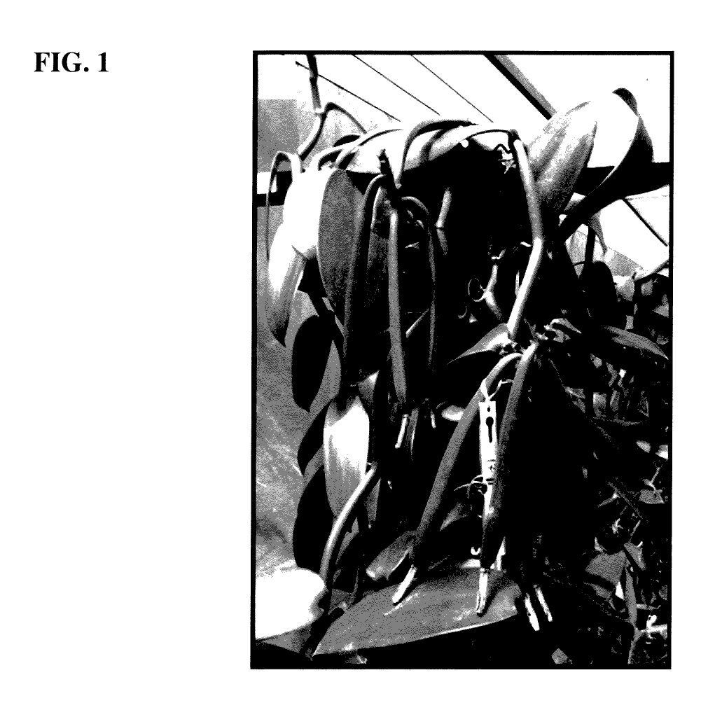

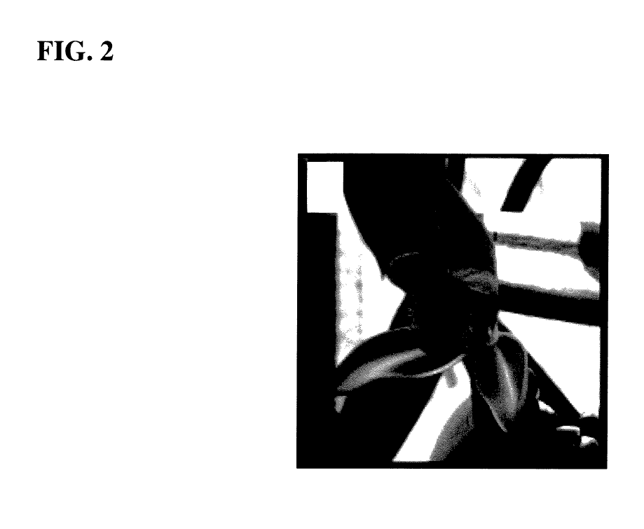

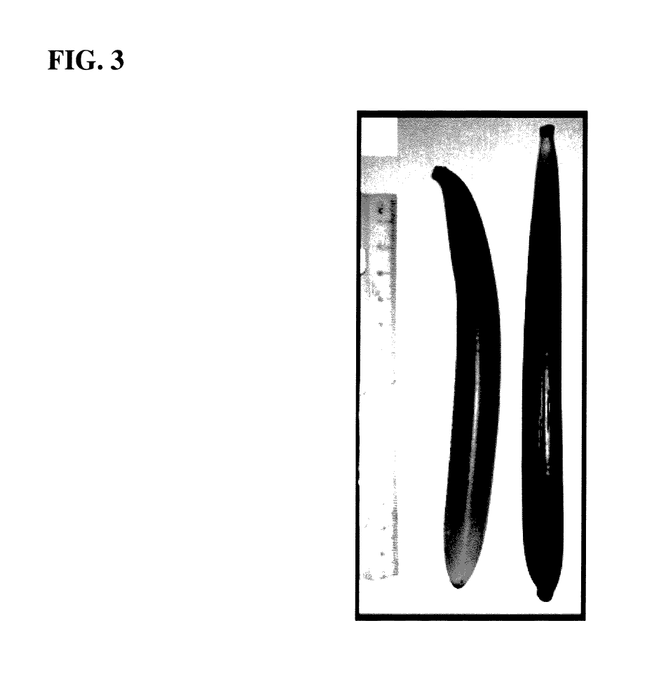

Vanilla variety ‘Handa’ is characterized by its aromatic fruit having a suitable vanillin content, as well as exhibiting superior agronomic and technologic properties as compared to ‘Ordinary’ vanilla, notably resistance to Fusarium oxysporum f. sp. radicis vanillae (Koyyappurath et al. 2015b), and indehiscence of the fruit. Genetic analysis using Amplified Fragment Length Polymorphism has been able to unequivocally distinguish the vanilla variety ‘Handa’ from all ‘Ordinary’ vanilla and many other V. planifolia varieties (Bory et al. 2008b). An image of the vines of vanilla variety ‘Handa’ bearing young fruit is presented in FIG. 1. An image of a flower of vanilla variety ‘Handa’ is presented in FIG. 2. An image of both green fruit and fully mature, non-dehiscent fruit of vanilla variety ‘Handa’ is presented in FIG. 3.

Table 2 below provides various morphological characteristics of vanilla variety ‘Handa’ as compared to those of ‘Ordinary’ vanilla.

TABLE 2
Morphological characteristics of ‘Handa’ as
compared to ‘Ordinary’ vanilla

| Characteristic | ‘Handa’ | ‘Ordinary’ |
|---|---|---|
| Leaf thickness | Thick | Medium |
| Leaf: intensity of green color | Dark | Medium |
| Fruit cross section | Circular | Broad ovate |

Table 3 below provides various quantitative characteristics of the fruit of vanilla variety ‘Flanda’ as compared to the fruit of ‘Ordinary’ vanilla.

TABLE 3
Quantitative Characteristics of ‘Handa’ fruit
as compared to ‘Ordinary’ vanilla

| | | | Relative |
|---|---|---|---|
| Characteristic | ‘Handa’ | ‘Ordinary’ | difference ⁽²⁾ |
| Weigh of a mature | 25.7 (+/−3)⁽¹⁾ | 21.1 (+/−2.94) | +22% |
| fruit (g) | | | |
| Length of a | 19 (+/−0.5) | 20.3 (+/−0.3) | −6% |
| mature fruit (cm) | | | |
| Percentage of | 0% | 98% | nd |
| dehiscent fruits | | | |
| Vanillin content | 2.51 (+/−0.5) | 3.71 (+/−0.24) | −32% |
| of a mature fruit⁽³⁾ | | | |
| (% dry weight) | | | |
| ParahydroxyBenz- | 0.07 (+/−0.06) | 0.08 (+/−0.03) | −13% |
| aldehyde content⁽³⁾ | | | |
| (% dry weight) | | | |
| ParahydroxyBenzyl | 0.2 (+/−0.08) | 0.15 (+/−0.02) | +33% |
| alcohol content⁽³⁾ | | | |
| (% dry weight) | | | |

(1) Average values measured at BRC VATEL, standard error in brackets.
(2) Calculated as follows : (Handa − Ordinary)/Ordinary
(3)Determined by HPLC analysis after enzymatic hydrolysis (Palama et al. 2011; Perez Silva et al. 2011)

Table 4 below provides a summary of the various advantageous characteristics of vanilla variety ‘Handa’ as compared to ‘Ordinary’ vanilla. These characteristics illustrate the advantages of using vanilla variety ‘Handa’ over ‘Ordinary’ vanilla for vanilla production.

TABLE 4
Advantageous characteristics of ‘Handa’ as
compared to ‘Ordinary’ vanilla

| | | | Advantage of ‘Handa’ over |
|---|---|---|---|
| Criteria | ‘Ordinary’ | ‘Handa’ | ‘Ordinary’ |
| Resistance to | Susceptible | Resistant | Increases productivity and |
| Fusarium root | | | longevity of plots. |
| rot | | | |
| Fruit weight | Medium | Heavy | Less fruits per Kg of vanilla = |
| | | | less manipulation per Kg. |
| Fruit | Dehiscent | Not | Allows fruit to reach full |
| dehiscence | | dehiscent | maturity in the field, facilitate |
| | | | vanilla curing, can compensate |
| | | | lower vanillin content. |

### REFERENCES

Bory S., Grisoni, M., Duval, M.-F., and Besse, P. 2008a. Biodiversity and preservation of vanilla: present state of knowledge. Genetic Resources and Crop Evolution 55: 551-571.

Bory S., Lubinsky, P., Risterucci, A.-M., Noyer, J.-L., Grisoni, M., Duval, M.-F., and Besse, P. 2008b. Patterns of introduction and diversification of Vanilla planifolia (Orchidaceae) in Reunion island (Indian Ocean). American Journal of Botany 95: 805-815.

Castillo Martinez R., and Engleman, Mark E. 1993. Caracterizacion de dos tipos de Vanilla planifolia. Acta Botanica Mexicana 25: 49-59.

Delassus M. 1963. La lutte contre la fusariose du vanillier par les méthodes génétiques. L'agronomic tropicalc 18: 245-246.

Grisoni M., Anli Liachouroutu, A. K., Beson, E., Pierard, E., Liew, E. C. Y., and Alabouvette, C. Year. “Phytosanitary constraints on Bourbon vanilla: past and present situations and perspectives for control”, In: vanilla2009, D. H. Frenkel(eds)).

Hernandez Hernandez J. 2011. Vanilla diseases. In: D. Havkin-Frenkel & F. C. Belanger (eds). Handbook of Vanilla Science and Technology:26-39. Wiley-Blackwell Chichester (UK).

Koyyappurath S., Atuahiva, T., Le Guen, R., Batina, H., Le Squin, S., Gautheron, N., Edel Hermann, V., Peribe, J., Jahiel, M., Steinberg, C., Liew, E. C. Y., Alabouvette, C., Besse, P., Dron, M., Sache, I., Laval, V., and Grisoni, M. 2015. Fusarium oxysporum f. sp. radicis-vanillae is the causal agent of root and stem rot of vanilla. Plant Pathology: n/a-n/a. 10.1111/ppa.12445

Koyyappurath, S., Conéjéro, G., Dijoux, J. B., Lapeyre Montes, F., Jade, K., Chiroleu, F., Gatineau, F., Verdeil, J. L., Besse, P., and Grisoni, M. (2015b). Differential responses of vanilla accessions to root rot and colonization by Fusarium oxysporum f. sp. radicis-vanillae. Frontiers in Plant Science 6. doi: 10.3389/fpls.2015.01125

Minoo D., Nirmal Babu, K. , Ravindran, P. N. , and Peter, K. V. 2006. Interspecific hybridization in vanilla and molecular characterization of hybrids and selfed progenies using RAPD and AFLP markers. Scientia Horticulturae. 108: 414-422.

Murashige T., and Skoog, F. 1962. A revised medium for rapid growth and bioassays with tobacco tissue cultures. Physiologia Plantarum 15: 473-497.

Palama T. L., Khatib, A., Choi, Y. H., Come, B., Fock, I., Verpoorte, R., and Kodja, H. 2011. Metabolic characterization of green pods from Vanilla planifolia accessions grown in La Réunion. Environmental and Experimental Botany 72: 258-265. DOI: 10.1016/j.envexpbot.2011.03.015

Pérez Silva A., Gunata, Z., Lepoutre, J.-P., and Odoux, E. 2011. New insight on the genesis and fate of odor-active compounds in vanilla beans (Vanilla planifolia G. Jackson) during traditional curing. Food Research International 44: 2930-2937.

Roux-Cuvelier M., and Grisoni, M. 2010. Conservation and Movement of Vanilla Germplasm. In: E. Odoux & M. Grisoni (eds). Vanilla:31-41. CRC Press, Boca Raton, FL (USA).

Soto Arenas M.A. 1999. Filogeografia y recursos genéticos de las vainillas de México [Online]. México. Available: http://www.conabio.gob.mx/institucion/proyectos/resultados/InfJ101.pdf [Accessed Marzo 31, 1999 2004].

Soto Arenas M. A. 2003. Vanilla. In: A. M. Pridgeon, P. J. Cribb, M. W. Chase & F. Ramunsen (eds). Genera orchidacearum, Vol 3, Orchidoideae (part 2):321-334. Oxford University Press.

Theis T., and Jimenez, T. A. 1957. A vanilla hybrid resistant to Fusarium root rot. Phytopathology 47: 578-581.

What is claimed is:

1. A new and distinct variety of vanilla designated ‘Handa’ as shown and described herein.
